(19) United States
(12) Patent Application Publication
(10) Pub. No.: US 2024/0180568 A1
(43) Pub. Date: Jun. 6, 2024

(54) THROMBOSIS REMOVAL DEVICE

(71) Applicant: HeMo Bioengineering Pte. Ltd., Singapore (SG)

(72) Inventors: Jack Chicheng Wang, Singapore (SG); Udayan Patel, San Jose, CA (US); Yuning Chen, Singapore (SG)

(21) Appl. No.: 18/527,742

(22) Filed: Dec. 4, 2023

Related U.S. Application Data
(60) Provisional application No. 63/429,730, filed on Dec. 2, 2022.

(51) Int. Cl. A61B 17/22; A61B 17/34

(52) U.S. Cl. CPC A61B 17/22012; A61B 17/3423; A61B 17/3462; A61B 17/3498; A61B 2017/22038; A61B 2017/22079; A61B 2017/22084; A61B 2017/2215

(57) ABSTRACT

The present disclosure relates to an endovascular medical device that assists the operator in removal of blood flow-obstructing clots from the vasculature utilizing a minimally invasive procedure.

### REFERENCED APPLICATIONS

The present application claims priority to U.S. Provisional Application Ser. No. 63/429,730 filed Dec. 2, 2022, which is incorporated herein by reference.

### THE FIELD OF DISCLOSURE

The present disclosure relates to an endovascular medical device that assists the operator in the removal of a blood flow-obstructing clot from the vasculature utilizing a minimally invasive procedure.

### BACKGROUND OF DISCLOSURE

It is often desirable to remove tissue from the body in a minimally invasive manner as possible as not to damage other tissue. For example, removal of tissue (e.g., blood clots, plaque, etc.) from the vasculature typically improves patient conditions and quality of life. Clinical data indicates that clot removal is beneficial or even necessary to improve outcomes. For example, in the peripheral vasculature, the removal of clots can reduce the need for amputation by 80 percent. The ultimate goal of any modality to treat these conditions of the arterial or venous system is to quickly, safely, and cost effectively remove the blockage or restore patency. This may be achieved by thrombus dissolution, fragmentation, thrombus aspiration or a combination of these methods.

Stenosis in a blood vessel is caused by the formation of plaque or thrombus. The current means of relieving or removing the stenosis is by angioplasty in which dilatation of the blood vessel is driven by the inflation of a balloon catheter at the area of the stenosis. However, not all stenosis vessels can be treated by angioplasty since many blockages are due to thrombus which may have formed at the location of atherosclerotic blockage or a thrombus emboli that was transported to the blockage site from other parts of the arterial system.

It is well known that the majority of the thrombus-containing lesions are not angiographically pre-identified as thrombotic. This is because the thrombus can be over shadowed by the necrotic core of a plaque. As such, it is common to understate the thrombus, while over identifying the calcium. In either situation, the best solution to treatment is typically to simply remove thrombus by thrombectomy or by simple suction.

When the necrotic cap of an atherosclerotic cap erodes or ruptures, the natural healing process involves the formation of a clot. The clot or thrombus that is formed adheres to the vessel wall at the site of atherosclerosis. The fresh thrombus is soft and over time becomes organized and fibrous. The fresh clot is easier to remove than an aged and organized clot. It has been reported that there is a difference in the feel when crossing the thrombus with a guide wire, thereby providing some indication as to extent and organization of the clot. Based on the extent to which the clot has believed to have organized, an appropriate treatment of the clot is selected. In general, a soft clot is removed easily by using an aspiration catheter while; an organized clot may optionally require lysis maceration of the clot followed by suction or use of a stent retriever. When the clot is extremely organized and hardened, a more drastic means of maceration, such as rotational or vibrational thrombectomy devices, are used.

Catheter-directed thrombectomy and thrombolysis are commonly perceived to be less traumatic, but less likely to decrease the morbidity and mortality associated with conventional surgical techniques because the current thrombectomy techniques can lead to downstream thrombus fragments (emboli) which can lead to blockage of small diameter branches.

Further disadvantages to current thrombectomy devices include the difficulty of keeping the clot in the space during the capture and removal. Some clots are adhered to the wall while others are simply emboli floated from a different parts of the vasculature. The degree of adherence varies with the extent of clot formation; therefore, its removal with current devices is typically never complete.

During the thrombectomy procedure, portions of a clot can dislodge and flow further downstream and cause blockages in smaller vessels. Also, some clots are organized and adhere in place or emboli and are typically fibrous and hardened. Current devices (known as stent retriever devices) are configured to capture such organized and hardened clots and then drag such clot along the length of the vessel until the clot can be removed from the vessel access point. This path of dragging the clot is usually long and tortuous and has high potential of damaging the vessel during retrieval and removal of the clot from the vessel.

Present day thrombectomy devices do not have the capability to aspirate. A few of the present devices can perform aspiration from the proximal side of the clot. In these devices, the tip is distal to the aspiration opening; hence, the emboli caused by the distal tip has a potential to float downstream instead of being aspirated. This disadvantage is the main drawback and can lead to post-thrombectomy adverse events due to embolic particles flowing downstream and blocking smaller blood vessels. An embolic filter may be deployed prior to such a procedure; however, such a filter is limited in size to capture only smaller quantities of clot. Eventually, the basket of the filter occludes the inside of the lumen, thereby preventing the concurrent use with a positioning and/or supporting guidewire. Depending on the stiffness of the tissue (e.g., clot) being removed from the vessel, retrieval of the basket often collapses the distal end of the catheter and prevents its use. The basket can then be difficult to pull into the catheter, particularly when holding a clot. The retrieval of the clot can thus result in sheering the clot out of the capture basket. Also, the basket generally is preloaded into the distal end of the catheter prior to insertion into the vessel, and preloading may be both difficult and time consuming, and may risk disrupting the device prior to deployment.

In view of the current state of thrombectomy devices, there is a need for a thrombectomy device, and particularly a mechanical thrombectomy device, that can be more effective in removing tissue such as clots from within a body. The thrombectomy device in accordance with the present disclosure overcomes the drawbacks of the present-day devices while ensuring the full capture and removal of the clot from the vasculature without causing injury to the healthy vessels. Described herein are apparatuses (devices, systems and kit) and methods of using thrombectomy devices to address the needs and problems discussed above.

### SUMMARY OF DISCLOSURE

In accordance with one non-limiting aspect of the present disclosure, there is provided an endovascular medical device to assist the operator in removal of blood flow-obstructing clot from the vasculature by utilizing a minimally invasive procedure.

In accordance with another and/or alternative non-limiting aspect of the present disclosure, there is provided an endovascular medical device that is directed to, but not limited to, the treatment of a bodily passageway that is obstructed by a tissue mass, such as a clot or thrombus. The endovascular medical device comprises a) a capture tube (e.g., a hollow outer tubular body), b) a second tubular body that is slidably positioned within the capture tube and wherein the second tubular body extends beyond both the proximal and distal ends of the capture tube, c) an optional third tubular body (e.g., inner tube) that is slidably positioned within the second tubular body and wherein a distal region of the third tubular body is located at or near the distal end of the second tubular body and can move outwardly from the distal end of the second tubular body, and d) a self-expandable stent or self-expandable porous structure.

In accordance with another and/or alternative non-limiting aspect of the present disclosure, the proximal end of the capture tube can optionally be attached to a y-arm luer. The y-arm luer can be connected to an aspiration device. The aspiration device can be configured to draw fluid and/or materials (e.g., clot fragments, etc.) from the distal end or distal region of the capture tube, the second tubular body, and/or the optional third tubular body and toward the proximal end or proximal region of the capture tube, the second tubular body, and/or the optional third tubular body. In one non-limiting embodiment, the y-arm luer can optionally have an integral hemostasis valve or a septum. Optionally, the y-arm can be attached to an aspiration device. A guide wire can optionally be threaded through the distal end of the device which exits out through the luer at the proximal end. Optionally, the luer can be attached to an aspiration device. Optionally, the luer can be attached to an infusion device to infuse saline, therapeutic agent, or lytic drug.

In accordance with another and/or alternative non-limiting aspect of the present disclosure, the self-expanding stent or self-expandable porous structure is optionally formed of a nickel titanium alloy (e.g., Nitinol™). As can be appreciated, other shape memory material can be used to partially or fully form the self-expanding stent or self-expandable porous structure (e.g., copper, zinc and aluminum (Cu—Zn—Al) alloys; copper, aluminum and nickel (Cu—Al—Ni); iron, manganese and silicon (Fe—Mn—Si) alloys, shape-memory polymers (SMPs), etc.). The self-expanding stent or self-expandable porous structure is not limited in configuration.

In accordance with another and/or alternative non-limiting aspect of the present disclosure, the proximal end of the self-expanding stent or self-expandable porous structure is optionally non-slidably connected to the second tubular body (e.g., top surface of the second tubular body, etc.). The type of connection arrangement is non-limiting (e.g., adhesive, solder, clamp, etc.).

In accordance with another and/or alternative non-limiting aspect of the present disclosure, the third tubular member is optionally absent in the endovascular medical device, and the proximal end of the self-expanding stent or self-expandable porous structure is optionally non-slidably connected to the second tubular member. The distal end of the self-expanding stent or self-expandable porous structure is connected to slidable ring that is slidably positioned about a distal region of the second tubular body. The slidable ring is configured to move along the outer surface of the second tubular body as the self-expanding stent or self-expandable porous structure moves between the expanded and contracted positions. For example, as the distal portion of the second tubular body including the self-expanding stent or self-expandable porous structure is drawn within the cavity of the capture tube, the self-expanding stent or self-expandable porous structure is forced to contract and thereby causes the slidable ring to move toward the distal end of the second tubular body. As the distal portion of the second tubular body including the self-expanding stent or self-expandable porous structure is extended outwardly from the cavity of the capture tube, the self-expanding stent or self-expandable porous structure can expand and thereby cause the slidable ring to move toward the proximal end of the second tubular body.

In accordance with another and/or alternative non-limiting aspect of the present disclosure, the endovascular medical device is optionally absent the third tubular member, and the region of the outer surface of the second tubular body that is located below the self-expanding stent or self-expandable porous structure includes one or more openings. The one or more openings or holes are configured to enable fluid and other material (e.g., clot fragments, etc.) to be drawn into and through the one or more openings or holes and into the interior cavity of the second tubular body when the aspiration device is activated. The fluid and other material drawn into the interior cavity of the second tubular body proceeds to flow toward the proximal end of the second tubular body and is then drawn out of the endovascular medical device by the aspiration device. The one or more openings or holes in the outer surface of the second tubular body can be optionally positioned proximal to the slidable ring and/or distal to the non-slidably connection at the proximal end of the self-expanding stent or self-expandable porous structure. The number and/or size of the one or more openings or holes are non-limiting.

In accordance with another and/or alternative non-limiting aspect of the present disclosure, the endovascular medical device is optionally absent the third tubular member, and the front end of the second tubular body can optionally be capped closed and/or include a reduced sized opening that allows a guide wire to be passed therethrough.

In accordance with another and/or alternative non-limiting aspect of the present disclosure, the endovascular medical device optionally includes the third tubular member. A proximal portion of the third tubular member is located in the cavity of the second tubular body. A distal portion of the third tubular member extends outwardly from the distal end of the second tubular body, and a distal end of the self-expanding stent or self-expandable porous structure is non-slidably connected to a distal portion or distal end of the third tubular member. The proximal end of the self-expanding stent or self-expandable porous structure is non-slidably connected to an outer surface of the second tubular member. The third tubular member is configured to move within the interior cavity of the second tubular body as the self-expanding stent or self-expandable porous structure moves between the expanded and contracted positions. For example, as the distal portion of the second tubular body connected to the distal end of the self-expanding stent or self-expandable porous structure is drawn within the cavity of the capture tube, the self-expanding stent or self-expandable porous structure is forced to contract and thereby causes the third tubular member to move toward the distal end of the second tubular body. As the distal portion of the second tubular body connected to the distal end of the self-expanding stent or self-expandable porous structure is extended outwardly from the cavity of the capture tube, the self-expanding stent or self-expandable porous structure can expand and thereby causes the third tubular member to move toward the proximal end of the second tubular body.

In accordance with another and/or alternative non-limiting aspect of the present disclosure, the endovascular medical device optionally includes the third tubular member. The region of the outer surface of the third tubular body located below the self-expanding stent or self-expandable porous structure includes one or more openings. The one or more openings or holes are configured to enable fluid and other material (e.g., clot fragments, etc.) to be drawn into and through the one or more openings or holes and into the interior cavity of the third tubular body when the aspiration device is activated. The fluid and other material drawn into the interior cavity of the third tubular body proceeds to flow toward the proximal end of the third tubular body and is then drawn out of the endovascular medical device by the aspiration device. When the third tubular body does not extend the full longitudinal length of the second tubular body, the fluid and other material that exits the proximal end of the third tubular body can then flow into the cavity of the second tubular body, and thereafter be drawn out of the endovascular medical device by the aspiration device. The one or more openings or holes in the outer surface of the third tubular body can be optionally positioned proximal to the location where the self-expanding stent or self-expandable porous structure is connected to the third tubular body. The second tubular member can optionally include one or more openings or holes in the outer surface of the second tubular body at a location w is proximal and/or distal to the location where the self-expanding stent or self-expandable porous structure is connected to the second tubular body. The number and/or size of the one or more openings or holes are non-limiting.

In accordance with another and/or alternative non-limiting aspect of the present disclosure, the endovascular medical device optionally includes the third tubular member. The front end of the third tubular body can optionally be capped closed and/or include a reduced sized opening that allows a guide wire to be passed therethrough.

In accordance with another and/or alternative non-limiting aspect of the present disclosure, one or more of the components of the endovascular medical device can be formed of a variety of materials (e.g., Nitinol™, stainless steel, cobalt-chromium alloy, refractory metals and refractory metal alloys, polyester, nylon, urethane, polymeric materials, etc.).

In accordance with another and/or alternative non-limiting aspect of the present disclosure, the second tubular member and/or optionally the self-expanding stent or self-expandable porous structure can be configured to be reshaped, collapsed, or folded when retractable within the distal end of the capture tube. Upon removal from the distal end of the capture tube, the self-expanding stent or self-expandable porous structure is capable of expanding partially or fully to its original shape.

In accordance with another and/or alternative non-limiting aspect of the present disclosure, the second and/or optionally third tubular body can optionally have a large opening at the proximal end to accept clot fragments. The second and/or optionally third tubular body can optionally have a large opening at both ends. The second and/or optionally third tubular body can optionally be partially or fully formed of a mesh or braid or a coiled material.

In accordance with another and/or alternative non-limiting aspect of the present disclosure, the proximal end of the second tubular body can optionally be fluidly connected to a vacuum suction device via the detachable luer for aspiration.

In accordance with another and/or alternative non-limiting aspect of the present disclosure, a guide wire can optionally be introduced into the distal end of the second tubular body and passed through the distal end of the second tubular body, and the y-arm luer can optionally be simultaneously connected to a vacuum source for aspiration.

In accordance with another and/or alternative non-limiting aspect of the present disclosure, there is a method for using the endovascular medical device comprising a) positioning the self-expanding stent or self-expandable porous structure fully or substantially inside the capture tube such that the self-expanding stent or self-expandable porous structure is in the unexpanded position; b) inserting a guidewire in the body passageway until the distal end of the guidewire is at least closely adjacent to the clot in the body passageway (e.g., within a few centimeters of the clot, at the proximal end of the clot, in the clot, beyond the distal end of the clot, etc.); c) introducing the endovascular medical device in the body passageway via the guidewire until the distal end of the endovascular medical device is at least closely adjacent to the proximal end of the clot; d) optionally removing the guidewire from the body passageway; e) activating an aspiration device; f) deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway; g) moving one or more tubular bodies of the endovascular medical device to cause the at least partially expanded self-expanding stent or self-expandable porous structure to contact a portion of the clot and cause at least a portion of the clot to fracture and/or release from the body passageway and be drawn by the aspiration device into at least one of the tubular bodies (e.g., drawn into the distal end of at least one of the tubular bodies, drawing into and through one or more of the openings or holes in the surface of at least one of the tubular bodies, etc.); h) at least partially or fully retracting the at least partially expanded self-expanding stent or self-expandable porous structure into the cavity of the capture tube to cause the self-expanding stent or self-expandable porous structure to partially or fully contract, and wherein fractured clots on the self-expanding stent or self-expandable porous structure are drawn from the self-expanding stent or self-expandable porous structure toward the proximal end of the capture tube by the aspiration device, and wherein the retracting of the self-expanding stent or self-expandable porous structure into the cavity of the capture tube optionally causes the self-expanding stent or self-expandable porous structure to grate against and at least partially fracture and/or remove the clot from the body passageway; i) optionally repeating steps f), g) and h) until the distal end of one or more of the tubular bodies are positioned distally to the distal end of the clot; j) optionally deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway and moving the endovascular medical device to cause the at least partially expanded self-expanding stent or self-expandable porous structure to move across the former region of the clot to partially or fully remove remaining portions of the clot from the body passageway that remained in the body passageway after the completion of steps f), g), h) and i); k) at least partially or fully retracting the at least partially expanded self-expanding stent or self-expandable porous structure into the cavity of the capture tube to cause the self-expanding stent or self-expandable porous structure to partially or fully contract, and wherein fractured clots on the self-expanding stent or self-expandable porous structure are drawn from the self-expanding stent or self-expandable porous structure toward the proximal end of the capture tube by the aspiration device, and l) remove the endovascular medical device from the body passageway. As can be appreciated, the above method can be modified by the elimination of the use of the self-expanding stent or self-expandable porous structure and the steps that are associated with the self-expanding stent or self-expandable porous structure. In one non-limiting embodiment, the one or more tubular bodies that are connected to the self-expanding stent or self-expandable porous structure can be caused to collapse and expand during the clot removal procedure. In another non-limiting embodiment, the one or more tubular bodies that include one or more openings or holes in the top surface of the tubular body cause further fracturing and/or grating of the clot as the clot portions more through the one or more openings or holes. In another non-limiting embodiment, the aspiration device causes any loose clot or thrombus located at the distal end and/or proximal end of the clot to be drawn into the interior of the capture tube; thus, preventing any particulates from floating downstream from the clot.

In accordance with another and/or alternative non-limiting aspect of the present disclosure, the capture tube can optionally be exchangeable. In such an arrangement, once the second and/or third tubular bodies are positioned at or closely to the proximal end of the clot, the capture tube can be retracted from the body passageway and another capture tube of generally a larger inner diameter is introduced to the proximal end of the clot. The alternate capture tube having the larger inner diameter has a better ability to recover the self-expanding stent or other type of self-expanding porous structure with large proportions of the clot.

In accordance with another and/or alternative non-limiting aspect of the present disclosure, the self-expanding stent or other type of self-expanding porous structure can cause portions of the clot to be grated and/or fractured as the self-expanding stent or other type of self-expanding porous structure is expanded and/or contracted in the body passageway.

In accordance with another and/or alternative non-limiting aspect of the present disclosure, the capture tube can optionally include a side opening for ease of exchange of the capture tube. The side opening can be located at any location between the distal end and the proximal end of the capture tube. The side hole or opening can optionally include an elastic septum that allows the second tubular body to pass through, but seals around the second tubular body to prevent blood flow across the side hole or opening during aspiration.

In accordance with another and/or alternative non-limiting aspect of the present disclosure, the distal end of the capture tube is optionally expandable to more easily accept the self-expanding stent or other type of self-expanding porous structure that contains portions of the clot. In one non-limiting embodiment, once the self-expanding stent or other type of self-expanding porous structure is retracted within the expanded distal end of the capture tube, the expanded distal end of the capture tube can be collapsed.

In accordance with another and/or alternative non-limiting aspect of the present disclosure, a guide sheath and guide wire self-expanding stent or self-expandable porous structure positions the distal end of the endovascular medical device at a location that is distal to the distal end of the clot; thereafter, the self-expanding stent or other type of self-expanding porous structure can be deployed and partially or fully expanded, and thereafter the deployed self-expanding stent or other type of self-expanding porous structure is moved toward the proximal end of the clot while the aspiration device is activated. During the movement of the self-expanding stent or other type of self-expanding porous structure toward the proximal end of the clot, the self-expanding stent or other type of self-expanding porous structure can be retracted into the capture tube and then redeployed one or more times.

In accordance with another and/or alternative non-limiting aspect of the present disclosure, there is provided an endovascular medical device that is directed to, but not limited to, the treatment of a bodily passageway that is obstructed by a tissue mass such as a clot or thrombus. The endovascular medical device comprises a catheter, a device shaft that is at least partially positioned in the cavity of the catheter and runs at least partially the length of the catheter, and a fragmenter that is positioned at the front end or front end portion of the device shaft. The catheter can be formed of one or more hollow tubular bodies. One of the tubes of the catheter can optionally be a guide wire tube. The proximal end of the catheter is configured to be attached to a handle. The length, shape, and/or materials of the catheter are non-limiting. The configuration of the handle is non-limiting. The handle is configured for use by a medical practitioner to insert and/or orient the catheter in the body passageway of the patient. In one non-limiting embodiment, the device shaft runs 70-100% (and all values and ranges therebetween) of the longitudinal length of the catheter located in the body passageway of the patient. In another non-limiting embodiment, the device shaft can be configured to cause the fragmenter to extend beyond a portion (e.g., 10-99% and all values and ranges therebetween) or all of the distal end of the catheter during use of the fragmenter when removing the clot from the boy passageway. In another non-limiting embodiment, the device shaft extends rearwardly of the proximal end of the catheter and is connected to the handle. The fragmenter is configured to facilitate in the removal of a clot in the body passageway of a patient.

In accordance with another and/or alternative non-limiting aspect of the present disclosure, the fragmenter includes a main housing and a plurality of non-smooth surface structures that extend outwardly and/or inwardly from the top surface of the main housing. The number and shape of the non-smooth surface structures are non-limiting. In one non-limiting embodiment, all of the non-smooth surface structures are oriented symmetrically with one another. In another non-limiting embodiment, a plurality of the non-smooth surface structures are oriented non-symmetrically with one another. In another non-limiting embodiment, the main housing has a generally cylindrical shape; however, other shapes can be used.

In accordance with another and/or alternative non-limiting aspect of the present disclosure, the device shaft is interconnected to the handle to enable the device shaft to be reciprocated and/or rotated in the catheter to thereby cause the reciprocation and/or rotation of the fragmenter in the body passageway. The movement of the fragmenter relative to the clot in the body passageway causes one or more of the non-smooth surface structures to contact the clot to thereby scrap off or fragment a portion or all of the clot in the body passageway. In one non-limiting embodiment, the handle includes one or more levers, knobs, slides, buttons, etc., that can be used by a medical practitioner to cause the device shaft to reciprocate and/or rotate during a clot removal procedure. In another non-limiting embodiment, the proximal end or end portion of the device shaft can be connected to a toothed cam in the handle such that the device shaft can be rotated as well as reciprocated along the longitudinal axis of the catheter. As can be appreciated, other or additional arrangement can be used to rotate and/or reciprocate the device shaft.

In accordance with another and/or alternative non-limiting aspect of the present disclosure, the device shaft can include a non-linear profile (e.g., wavy profile, curved profile, etc.) at the distal region of the device shaft. Such a non-linear device shaft profile causes the fragmenter to wobble in the body passageway to facilitate in causing the non-smooth surface structures to contact the clot in the body passageway.

In accordance with another and/or alternative non-limiting aspect of the present disclosure, the fragmenter can optionally include a hollow cavity and one or more surface openings that are fluidly connected to the hollow cavity. Such a hollow cavity and one or more surface openings draws fragments of the clot that have been removed from the clot in the body passageway as the non-smooth surface structures of the fragmenter contact the clot into the hollow cavity.

In accordance with another and/or alternative non-limiting aspect of the present disclosure, an aspirator system is used to draw into the catheter fragments of the clot that have been removed from the clot in the body passageway as the non-smooth surface structures of the fragmenter contact the clot. In one non-limiting embodiment, the handle (e.g., proximal end of the handle, etc.) can optionally include or be connected to a y-arm luer. In one non-limiting arrangement, the side arm of the y-arm luer protrudes out of the handle to be connected to an aspiration device. The straight arm of the y-arm luer can optionally have a hemotatsis valve through which the proximal end of the device shaft proximally exits such that the hemostasis valve does not excessively obstruct or otherwise interfere with the rotation and/or reciprocation of the device shaft.

In accordance with another and/or alternative non-limiting aspect of the present disclosure, the device shaft can be configured such that the distal end of the fragmenter can be located at the distal end of the catheter prior to the reciprocation and/or rotation of the fragmenter.

In accordance with another and/or alternative non-limiting aspect of the present disclosure, there is provided a method for the partial or full removal of a clot from a body passageway. The method can optionally be a minimally invasive intravascular procedure for the partial or full removal of a clot from a body passageway. The method includes a) navigating a guide wire to the treatment site within the vasculature, b) inserting the distal end or distal portion of the catheter on the proximal end of the guide wire (e.g., guide wire inserted into the short tube of catheter, etc.), c) moving the catheter over the guide wire until the distal end of the catheter is located at the treatment site, d) connecting an aspiration system to the aspiration port on the handle and/or catheter; e) activating the aspiration system to cause fluid to be drawn from the distal portion of the catheter to the proximal portion of the catheter, f) moving the distal end of the catheter to the proximal end of the clot if not already positioned at the clot, g) causing the fragmenter to be reciprocated and/or rotated and to engage at least a portion of the clot to cause partial or full fragmentation and/or removal of the clot from the vasculator, and h) removing the catheter, device shaft, and fragmenter from the vasculaturer once the desire amount of the clot has been partially or fully removed from the vasculature. In one non-limiting embodiment, when the fragmenter is caused to reciprocate, all or a portion (e.g., 10-99% and all values and ranges therebetween) extends forwardly of the distal end of the catheter to enable at least a portion of the fragmenter to contact the clot in the body passageway. In one non-limiting configuration, when the fragmenter is in the initial position prior to reciprocation, all or a majority (e.g., 60-99% and all values and ranges therebetween) is positioned within the catheter. When the fragmenter is the fully extended position during reciprocation, all or a majority (e.g., 60-99% and all values and ranges therebetween) of the fragmenter is positioned forwardly of the distal end of the catheter. In one non-limiting configuration, when a lever on the handle is squeezed or otherwise activated, such activation of the handle causes the fragmenter to move forwardly beyond the catheter distal end or tip of the catheter (and optionally also rotate) and into the clot. The movement of the fragmenter into all or a portion of the clot and the combined optional rotational movement of the fragmenter causes at least a portion of the clot to fragment, after which one or more fragmented clot portions are aspirated into the distal end of the catheter and drawn toward the proximal end of the catheter. When the handle is released or the fragmenter is otherwise caused to be withdrawn into the catheter, the retraction of the fragmenter can cause further fragmentation of the clot, after which one or more fragmented clot portions are aspirated into the distal end of the catheter and drawn toward the proximal end of the catheter. Also, when the fragmenter is caused to be withdrawn into the catheter, the fragmenter can optionally be caused to rotate in the same or opposite direction as the direction of rotation when the fragmenter was being extended from the catheter. The optional rotation of the fragmenter as the fragmenter is drawn into the catheter can also result in the further fragmentation of the clot, after which one or more fragmented clot portions are aspirated into the distal end of the catheter and drawn toward the proximal end of the catheter. In another non-limiting embodiment, the fragmenter can optionally be caused to be reciprocated and/or rotated by a motorized mechanism. In another non-limiting embodiment, the method of clot removal can optionally include the movement of the catheter forwardly after a portion of a clot has been removed to further remove additional clot from the body passageway. When the catheter is optionally moved forwardly, the above described method steps for clot removal can be repeated. As such, the moving of the catheter forwardly one or more times partially or fully removes the clot along the full length of a long clot. The method in accordance with the present disclosure includes the use of manual movement of the catheter and fragmenter by the medical practitioner to allow for improved control over the amounts of clots to be removed and from the treatment area during thrombectomy.

In accordance with another and/or alternative non-limiting aspect of the present disclosure, the guide wire can optionally be retracted once the distal tip of the catheter is positioned at or in close proximity to the clot.

In accordance with another and/or alternative non-limiting aspect of the present disclosure, the distal tip of the catheter can optionally be configured to radially expand to form a funnel shaped opening for better capture of the fragments of the clot produced by the contact of the fragmenter with the clot. In one non-limiting embodiment, the distal end of the catheter body is configured to flare out in a concave or convex shape.

In accordance with another and/or alternative non-limiting aspect of the present disclosure, there is provided an endovascular medical device that is directed to, but not limited to, the treatment of a bodily passageway that is obstructed by tissue mass such as a clot or thrombus.

In accordance with another and/or alternative non-limiting aspect of the present disclosure, the endovascular medical device includes a) a catheter that includes first, second, and third tubular structures; wherein said first and third tubular structures are slidably movable with respect to one another; and wherein the second and third tubular structure is positioned in the longitudinal cavity of the first tubular structure, and the third tubular structure is positioned in the longitudinal cavity of the second tubular structure, and b) a clot engagement material (e.g., wires, threads, mesh material, braided material). When the clot engagement material is a mesh or braided material, the clot engagement material can optionally be folded (e.g., folded edge-to-edge, etc.) to form an incomplete tubular structure within the first tubular structure. The third tubular structure has a proximal and a distal end, wherein the distal end or distal end portion is connected to an end or end portion of the clot engagement material. In one non-limiting configuration, the clot engagement material can be partially or fully wrapped around the outer surface of the distal portion of the third or inner tubular structure. The second or intermediate tubular structure is positioned about the third or inner tubular structure. Prior to deployment of the clot engagement material, the distal ends of the second and third tubular structures are aligned or substantially aligned. The clot engagement material can be inverted proximally over the distal end portion of the second tubular structure. The first or outer tubular structure is placed over the inverted clot engagement material. The distal end of the first tubular structure, prior to deployment of the clot engagement material, is also aligned or substantially aligned with the distal ends of the second and third tubular structures. Such an arrangement forms a structure in which the inverted clot engagement material lies in-between the first tubular structure and the second tubular structure and the separation slit of the clot engagement material runs longitudinally along a portion or all of the longitudinal length of the three tubular structures. The clot engagement material that is wrapped around the second tubular structure is non-limiting in longitudinal length.

In accordance with another and/or alternative non-limiting aspect of the present disclosure, the proximal portion of the first tubular structure includes a y-arm luer with an optional hemostasis valve. In one non-limiting embodiment, the y-arm is fluidly connected or interconnected to the proximal end portion of the second tubular structure. An aspirator system to draw fluid into the catheter can be connected to the y-arm luer. In another non-limiting embodiment, the third tubular structure optionally protrudes proximally through the hemostasis valve. A luer can optionally be attached to the proximal end or end region of the third tubular structure and be located proximal to the y-arm connected to the second tubular structure.

In accordance with another and/or alternative non-limiting aspect of the present disclosure, the first, second, and third tubular structures are aligned or substantially aligned with one another along the longitudinal axis of the catheter.

In accordance with another and/or alternative non-limiting aspect of the present disclosure, the distal ends of the third tubular structure and the clot engagement material are distal or located forwardly to the distal edge of the second tubular structure. The second tubular structure and the clot engagement material can optionally be bonded to each other.

In accordance with another and/or alternative non-limiting aspect of the present disclosure, there is method for removing a clot using the endovascular medical device wherein a) the first tubular structure is navigated over a guide wire to the treatment site and the distal end of the first tubular structure is placed at or closely adjacent to the clot in the body passageway, and b) the third tubular structure is then pulled toward the proximal end of the first tubular structure and at the same time the distal end of the first tubular structure is moved toward and into the clot. The opposite movements of the first and third tubular structures cause the clot engagement material to move distally over the top of the second tubular structure while the clot engagement material moves proximally inside the second tubular structure. Such movement of the clot engagement material along the top surface of the second tubular structure and then into the interior of the second tubular structure causes the clot that engages and is captured by the moving clot engagement material as the clot engagement material moves toward the distal end of the second tubular structure and then about the end of the second tubular structure, to be then pulled within the interior of the second tubular structure as the third tubular structure is retracted within the second tubular structure. While the first and third tubular structure are moved in opposite directions to one another, the aspiration system can optionally draw the clot into the interior of the second tubular structure that has engaged the clot engagement material as the clot engagement material is drawn into the interior of the second tubular structure.

In accordance with another and/or alternative non-limiting aspect of the present disclosure, the clot engagement material can be configured such that the longitudinal length of the clot engagement material is at least two times (e.g., 2-100 times and all values and ranges therebetween) the longitudinal length of the clot so that as the clot engagement material is pulled about the distal end of the second tubular structure during the removal of the clot from the body passageway, the length of clot engagement material will be more sufficient to fully remove the clot without having to reload the catheter with a new clot engagement material during the clot removal procedure. Such a configuration is a significant improvement over prior art clot removal devices.

In accordance with another and/or alternative non-limiting aspect of the present disclosure, the distal end portion of the second tubular structure can be optionally expanded to cause improved engagement of the clot engagement material with the clot as the clot engagement material moves about the distal end of the second tubular structure.

In accordance with another and/or alternative non-limiting aspect of the present disclosure, the distal edge of the first tubular structure can optionally be connected to the distal edge of the third tubular structure. In such an arrangement, the first tubular structure is formed of a soft flexible material that allows the first tubular structure to collapse over itself and invert as the first tubular structure is pulled into the third tubular structure. Such inversion of the first tubular structure can result in the pulling of the clot with the first tubular structure into the interior of the third tubular structure.

In accordance with another and/or alternative non-limiting aspect of the present disclosure, two or more tapes, strands, braids, or mesh of the clot engagement material can optionally be fed through the distal end of the second tubular structure during the clot removal procedure.

In accordance with another and/or alternative non-limiting aspect of the present disclosure, the clot engagement material is fed from the interior of the second tubular structure, and travels about the distal end of the second tubular structure, and then moves along the top surface of the second tubular structure toward the proximal end of the second tubular structure. In such a non-limiting arrangement, the third tubular structure can be eliminated. Also, in such non-limiting arrangement, the distal portion of the second tubular structure can be configured to expand to cause improved engagement, grabbing, and/or fracturing of the clot engagement material with the clot as the clot engagement material moves about the distal end of the second tubular structure. When the endovascular medical device only includes first and second tubular structures, the method is altered in that a) the distal end of the first tubular structure is positioned past the clot; b) the aspiration system (when used) is activated prior to the pulling of the clot engagement material; c) the clot engagement material is pulled proximally along the outer surface of the second tubular structure such that the clot engagement material is pulled distally in the interior of the second tubular structure toward the distal end of the second tubular structure and then about the distal end of the second tubular structure and then along the outer of the second tubular structure toward the proximal end of the second tubular structure; and d) moving the distal end of the first tubular structure from the distal end of the clot to the proximal end of the clot while the clot removal material is drawn about the distal end of the second tubular structure. The distal end of the second tubular structure can optionally be caused to flare outwardly by pulling the clot engagement material about the distal end of the second tubular structure at a faster rate than the moving of the distal end of the first tubular structure along the clot. In one non-limiting embodiment, the clot engagement materials can optionally be inverted on itself when placed in the interior of the second tubular structure. In such an arrangement, as the clot engagement material is drawn toward the distal end of the second tubular structure, one layer of the clot engagement material is being pushed while other layer is pulled over itself, thereby causing the captured clot to move proximally and into the interior of the first tubular structure.

In accordance with another and/or alternative non-limiting aspect of the present disclosure, the distal edge or distal end portion of the second tubular structure is optionally cut (e.g., oblique cut, etc.) to form a larger opening at the distal end of the second tubular structure to a) improve contact with the clot during the removal of the clot, b) allow for easier movement of the clot engagement material as the clot engagement material and clot material are drawn into the interior of the second tubular structure, and/or c) allow for easier movement of the clot engagement material as the clot engagement material is drawn from the interior of the second tubular structure and about the outer surface of the second tubular structure.

One non-limiting object of the present disclosure is the provision of an endovascular medical device that assists the operator in the removal of a blood flow-obstructing clot from the vasculature.

Another non-limiting object of the present disclosure is the provision of an endovascular medical device that assists the operator in the removal of a blood flow-obstructing clot from the vasculature utilizing a minimally invasive procedure.

Another non-limiting object of the present disclosure is the provision of an endovascular medical device that includes an outer tubular catheter body, an inner tubular body that includes one or more fragmenters; at least one of the fragmenters is located at a distal end the inner tubular body and can be positionable within a distal end of the outer tubular catheter body when the inner tubular body is at least partially retracted in the outer tubular catheter body; the inner tubular body and the one or more fragmenters movable relative to the outer tubular catheter body by a hand-operated mechanical arrangement and/or by a motor arrangement; the outer tubular catheter body includes a luer connector.

Another non-limiting object of the present disclosure is the provision of an endovascular medical device wherein the inner tubular body and the one or more fragmenters are configured to be rotatable relative to the outer tubular catheter body.

Another non-limiting object of the present disclosure is the provision of an endovascular medical device wherein the inner tubular body and the one or more fragmenters are configured to reciprocate relative to the outer tubular catheter body.

Another non-limiting object of the present disclosure is the provision of an endovascular medical device wherein the inner tubular body and the one or more fragmenters are configured to vibrate relative to the outer tubular catheter body.

Another non-limiting object of the present disclosure is the provision of an endovascular medical device wherein the inner tubular body and the one or more fragmenters are configured to radiate electromagnetic waves relative to the outer tubular catheter body.

Another non-limiting object of the present disclosure is the provision of an endovascular medical device wherein a proximal end of the luer connector includes a hemostasis valve through which a proximal end the inner tubular body can extend outwardly therefrom.

Another non-limiting object of the present disclosure is the provision of an endovascular medical device wherein the inner tubular body is hollow and is configured to allow a guide wire to pass there through the inner tubular body.

Another non-limiting object of the present disclosure is the provision of an endovascular medical device wherein the inner tubular body is hollow and is configured to allow fluid to flow through the hollow tubular body; the fluid includes one or more of saline solution, water, clot dissolving thrombolytic agent, and/or anti-clotting agent.

Another non-limiting object of the present disclosure is the provision of an endovascular medical device wherein the fragmenter includes a non-smooth outer surface that includes one or more non-smooth surface structures; the one or more non-smooth surface structures include one or more structures selected from the group consisting of ribs, spikes, bumps, undulations, channels, slots, recesses, barbs, pins, blades, surface extensions, and fluted surface extensions.

Another non-limiting object of the present disclosure is the provision of an endovascular medical device wherein the inner tubular body is partially or fully formed of 1-16 wires.

Another non-limiting object of the present disclosure is the provision of an endovascular medical device wherein the inner tubular body is partially or fully formed of a plurality of wires that are braided together; the inner tubular body is flexible.

Another non-limiting object of the present disclosure is the provision of an endovascular medical device for the treatment of a bodily passageway that is obstructed by tissue mass such as a clot or thrombus; the endovascular medical device comprises a catheter, a device shaft at least partially positioned in the cavity of the catheter and running at least partially the length of the catheter, and a fragmenter positioned at the front end or front end portion of the device shaft.

Another non-limiting object of the present disclosure is the provision of an endovascular medical device further including a handle that is connected to a proximal portion of the catheter; the handle configured to cause a) rotation of the device shaft, and/or b) reciprocation of the device shaped along a longitudinal axis of the catheter.

Another non-limiting object of the present disclosure is the provision of an endovascular medical device wherein the fragmenter includes a main housing and a plurality of non-smooth surface structures that extend outwardly and/or inwardly from the top surface of the main housing.

Another non-limiting object of the present disclosure is the provision of an endovascular medical device wherein the device shaft includes a non-linear profile at a distal region of the device shaft.

Another non-limiting object of the present disclosure is the provision of an endovascular medical device further including an aspirator system that is configured to draw material into the distal end of the catheter.

Another non-limiting object of the present disclosure is the provision of an endovascular medical device wherein a distal end portion of the catheter is configured to radially expand to form a funnel-shaped opening.

Another non-limiting object of the present disclosure is the provision of an endovascular medical device for the treatment of a bodily passageway that is obstructed by tissue mass such as a clot or thrombus; the endovascular medical device comprising a) a catheter that includes first, second, and third tubular structures; the first and third tubular structures are slidably movable with respect to one another; and wherein the second and third tubular structure is positioned in the longitudinal cavity of the first tubular structure, and the third tubular structure is positioned in the longitudinal cavity of the second tubular structure, and b) a clot engagement material.

Another non-limiting object of the present disclosure is the provision of an endovascular medical device wherein the clot engagement material is a mesh or braided material.

Another non-limiting object of the present disclosure is the provision of an endovascular medical device that wherein the clot engagement material is folded edge-to-edge to form an incomplete tubular structure within the first tubular structure.

Another non-limiting object of the present disclosure is the provision of an endovascular medical device wherein a distal end or distal end portion of the third tubular structure is connected to an end or end portion of the clot engagement material.

Another non-limiting object of the present disclosure is the provision of an endovascular medical device wherein the second tubular structure is oriented relative to the first tubular structure to not move or substantially not move along the longitudinal axis of the first tubular structure.

Another non-limiting object of the present disclosure is the provision of an endovascular medical device wherein the second tubular structure is connected to an interior of the first tubular structure.

Another non-limiting object of the present disclosure is the provision of an endovascular medical device wherein the clot engagement material is partially or fully wrapped around an outer surface of at least a portion of the third tubular structure.

Another non-limiting object of the present disclosure is the provision of an endovascular medical device wherein a) the first tubular structure is navigated over a guide wire to a treatment site and a distal end of the first tubular structure is placed at or closely adjacent to a proximal end of a clot in a body passageway, and b) the third tubular structure is pulled toward a proximal end of the first tubular structure while a distal end of the first tubular structure is move toward and into the clot.

Another non-limiting object of the present disclosure is the provision of an endovascular medical device wherein the distal end portion of the second tubular structure is expandable to enable the distal end portion to flare outwardly.

Another non-limiting object of the present disclosure is the provision of an endovascular medical device wherein the distal edge of the first tubular structure is connected to a distal edge of the third tubular structure; the distal portion of the first tubular structure formed of a soft flexible material that allows the first tubular structure to collapse over itself and invert as the first tubular structure is pulled into the third tubular structure.

Another non-limiting object of the present disclosure is the provision of an endovascular medical device for the treatment of a bodily passageway that is obstructed by tissue mass such as a clot or thrombus; the endovascular medical device comprises a) a catheter that includes first and second tubular structures; and wherein the first and second tubular structures are not slidably movable with respect to one another; and wherein the second tubular structure is positioned in the longitudinal cavity of the first tubular structure, and b) a clot engagement material that is at least partially positioned in an interior cavity of the second tube; and wherein the clot engagement material is configured to be drawn from the interior cavity of the second tube, about a distal end of the second tube and between an outer surface of the second tube and an inner cavity surface of the first tube.

Another non-limiting object of the present disclosure is the provision of an endovascular medical device wherein the clot engagement material is a mesh or braided material.

Another non-limiting object of the present disclosure is the provision of an endovascular medical device wherein the clot engagement material is folded edge-to-edge to form an incomplete tubular structure within the first tubular structure.

Another non-limiting object of the present disclosure is the provision of an endovascular medical device wherein a tether wire is connected to a front end or end portion of the clot engagement material; the tether wire located between an outer surface of the second tube and an inner cavity surface of the first tube and extending to a proximal end of the first tube.

Another non-limiting object of the present disclosure is the provision of an endovascular medical device wherein the second tubular structure is oriented relative to the first tubular structure to not move or substantially not move along the longitudinal axis of the first tubular structure.

Another non-limiting object of the present disclosure is the provision of an endovascular medical device wherein the second tubular structure is connected to an interior of the first tubular structure.

Another non-limiting object of the present disclosure is the provision of an endovascular medical device wherein a) the first tubular structure is navigated over a guide wire to a treatment site and a distal end of the second tubular structure is placed beyond a distal end of a clot in a body passageway, b) an aspiration system is activated prior to pulling of the clot engagement material over an outer surface of the second tube, c) pulling the clot engagement material proximally along the outer surface of the second tubular structure such that the clot engagement material is pulled distally in the interior of the second tubular structure toward the distal end of the second tubular structure and then about a distal end of the second tubular structure and then along the outer of the second tubular structure toward a proximal end of the second tubular structure, and d) moving the distal end of the second tubular structure from a distal end of the clot to a proximal end of the clot while the clot removal material is drawn about the distal end of the second tubular structure.

Another non-limiting object of the present disclosure is the provision of an endovascular medical device wherein the distal end portion of the second tubular structure is expandable to enable the distal end portion to flare outwardly.

Another non-limiting object of the present disclosure is the provision of an endovascular medical device wherein the distal end portion of the second tubular structure is oblique cut to form a larger opening at the distal end of the second tubular structure.

Another non-limiting object of the present disclosure is the provision of an endovascular medical device for the treatment of a bodily passageway that is obstructed by tissue mass such as a clot or thrombus; the endovascular medical device comprises a) a capture tube, b) a second tubular body that is slidably positioned within the capture tube and wherein the second tubular body optionally extends beyond both a proximal and distal ends of the capture tube, c) an optional third tubular body that is slidably positioned within the second tubular body and wherein a distal region of the third tubular body is located at or near a distal end of the second tubular body and can move outwardly from the distal end of the second tubular body, and d) a self-expandable stent or self-expandable porous structure.

Another non-limiting object of the present disclosure is the provision of an endovascular medical device wherein a proximal end of the capture tube is attached to a y-arm luer, and herein the y-arm luer is connected to an aspiration device.

Another non-limiting object of the present disclosure is the provision of an endovascular medical device wherein the self-expanding stent or self-expandable porous structure is formed of a nickel-titanium alloy.

Another non-limiting object of the present disclosure is the provision of an endovascular medical device wherein a proximal end of the self-expanding stent or self-expandable porous structure is non-slidably connected to the second tubular body.

Another non-limiting object of the present disclosure is the provision of an endovascular medical device wherein the self-expanding stent or self-expandable porous structure is non-slidably connected at a proximal end of the self-expanding stent or self-expandable porous structure to the second tubular member, and a distal end of the self-expanding stent or self-expandable porous structure is connected to a slidable ring that is slidably positioned about a distal region of the second tubular body.

Another non-limiting object of the present disclosure is the provision of an endovascular medical device wherein a region of an outer surface of the second tubular body located below the self-expanding stent or self-expandable porous structure includes one or more openings.

Another non-limiting object of the present disclosure is the provision of an endovascular medical device wherein a proximal portion of the third tubular member is located in a cavity of the second tubular body and a distal portion of the third tubular member extends outwardly from a distal end of the second tubular body; and wherein a distal end of the self-expanding stent or self-expandable porous structure is non-slidably connected to a distal portion or distal end of the third tubular member, and a proximal end of the self-expanding stent or self-expandable porous structure is non-slidably connected to an outer surface of the second tubular member; and wherein the third tubular member is configured to move within an interior cavity of the second tubular body.

Another non-limiting object of the present disclosure is the provision of an endovascular medical device wherein a region of an outer surface of the third tubular body located below the self-expanding stent or self-expandable porous structure includes one or more openings.

Another non-limiting object of the present disclosure is the provision of an endovascular medical device wherein the capture tube can include a side opening; the side opening optionally includes an elastic septum that allows the second tubular body to pass therethrough, but will form a seal around the second tubular body.

Another non-limiting object of the present disclosure is the provision of method that includes the use of an endovascular medical device that includes the steps of a) positioning the self-expanding stent or self-expandable porous structure fully or substantially inside the capture tube such that the self-expanding stent or self-expandable porous structure is in an unexpanded position; b) inserting a guidewire in a body passageway until a distal end of the guidewire is at least closely adjacent to the clot in the body passageway; c) introducing the endovascular medical device in the body passageway via the guidewire until a distal end of the endovascular medical device is at least closely adjacent to the proximal end of the clot; d) optionally removing the guidewire from the body passageway; e) activating an aspiration device; f) deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway; g) moving one or more tubular bodies of the endovascular medical device to cause the at least partially expanded self-expanding stent or self-expandable porous structure to contact a portion of the clot and cause at least a portion of the clot to fracture and/or release from the body passageway and be drawn by the aspiration device into at least one of the tubular bodies; h) at least partially or fully retracting the at least partially expanded self-expanding stent or self-expandable porous structure into the cavity of the capture tube to cause the self-expanding stent or self-expandable porous structure to partially or fully contract, and wherein fractured clots on the self-expanding stent or self-expandable porous structure are drawn from the self-expanding stent or self-expandable porous structure toward the proximal end of the capture tube by the aspiration device, and wherein the retracting of the self-expanding stent or self-expandable porous structure into the cavity of the capture tube optionally causes the self-expanding stent or self-expandable porous structure to grate against and at least partially fracture and/or remove the clot from the body passageway; i) optionally repeating steps f), g) and h) until a distal end of one or more of the tubular bodies are positioned distally to a distal end of the clot; j) optionally deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway, and thereafter optionally moving the endovascular medical device to cause the at least partially expanded self-expanding stent or self-expandable porous structure to move across a former region of the clot to partially or fully remove remaining portions of the clot from the body passageway that remained in the body passageway after completion of steps f), g), h) and i); k) at least partially or fully retracting the at least partially expanded self-expanding stent or self-expandable porous structure into the cavity of the capture tube to cause the self-expanding stent or self-expandable porous structure to partially or fully contract, and wherein fractured clots on the self-expanding stent or self-expandable porous structure are optionally drawn from the self-expanding stent or self-expandable porous structure toward the proximal end of the capture tube by the aspiration device; and l) removing the endovascular medical device from the body passageway.

Another non-limiting object of the present disclosure is the provision of method that includes the use of an endovascular medical device that includes the steps of positioning a guide sheath and guide wire to a distal end of a clot, positioning a distal end of the endovascular medical device at a location that is distal to the distal end of the clot, causing the self-expanding stent or other type of self-expanding porous structure to be deployed and partially or fully expanded, moving the deployed and partially or fully expanded self-expanding stent or other type of self-expanding porous structure toward a proximal end of the clot while an aspiration device is activated.

These and other advantages of the present disclosure will become more apparent to those skilled in the art from a review of the description of the preferred embodiment and claims.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 is a side view of one non-limiting embodiment of the endovascular medical device that includes a self-expandable stent or self-expandable porous structure.

FIG. 2A is a side view of a portion of the endovascular medical device of FIG. 1 illustrating the self-expandable stent or self-expandable porous structure positioned on the second tubular body and connected at the proximal end to the second tubular body and at the distal end to a slidable ring.

FIG. 2B is a modification of the embodiment illustrated in FIG. 2A wherein in the proximal end of the second tubular body includes a luer that is optionally detachable.

FIG. 3A is another non-limiting embodiment illustrating a third tubular body that is moveable within the second tubular body and the self-expandable stent or self-expandable porous structure is connected at the distal end to the third tubular body and at the proximal end to the second tubular body.

FIG. 3B is a modification of the embodiment illustrated in FIG. 3A wherein the proximal end of the second tubular body includes a detachable luer.

FIG. 4 is a side view of a portion of the endovascular medical device of FIG. 1 illustrating the proximal end of the second tubular body including a luer that is optionally detachable that is optionally detachable.

FIG. 5 is a side view of the endovascular medical device of FIG. 1 illustrating the self-expandable stent or self-expandable porous structure positioned in the retracted or collapsed position and retracted in the interior of the capture tube.

FIG. 6 is a side view of another non-limiting embodiment of the endovascular medical device that includes a fragmenter.

FIG. 7 is a side view of a device shaft and fragmenter as illustrated in FIG. 6.

FIGS. 8A-8E are various non-limiting configurations of a fragmenter.

FIG. 9A is a side view of another non-limiting embodiment wherein a distal portion of the device shaft is non-linear.

FIG. 9B is a side view of the endovascular medical device of FIG. 1 that includes the device shaft of FIG. 9A.

FIG. 9C is a side view of the endovascular medical device of FIG. 1 wherein the catheter includes a guide tube.

FIG. 9D is a side view of the endovascular medical device of FIG. 1 wherein the FIGS. 10A and 10B are sectional side view of the distal portion of the catheter that illustrates that the distal end of the catheter can be optionally expanded to a convex or concave shape.

FIG. 11 is a side view of another non-limiting embodiment of an endovascular medical device in accordance with the present disclosure.

FIG. 12 is a side view of another non-limiting embodiment of the endovascular medical device of FIG. 11 wherein the distal portion of the second tubular structure is outwardly flared.

### DETAILED DESCRIPTION OF NON-LIMITED EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

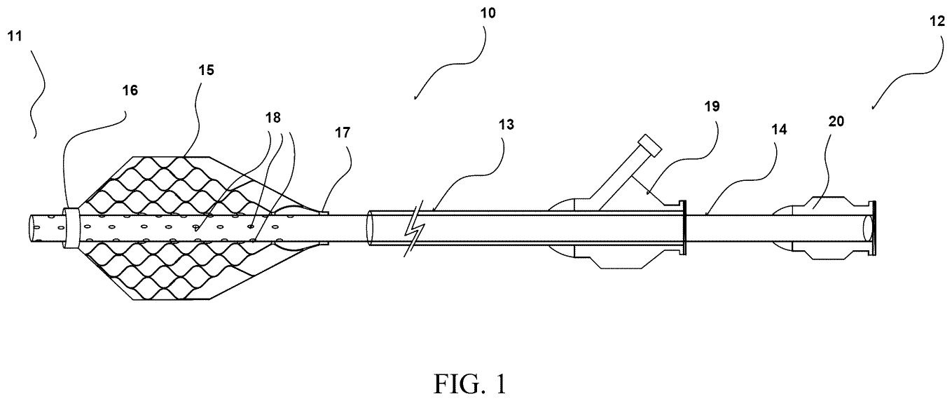

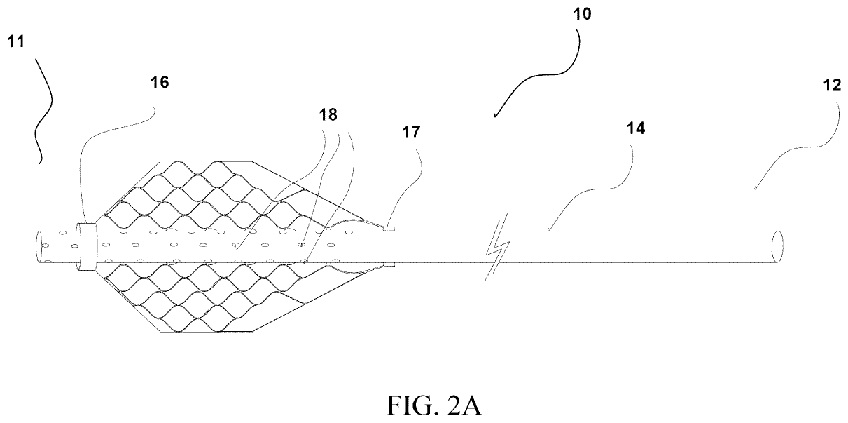

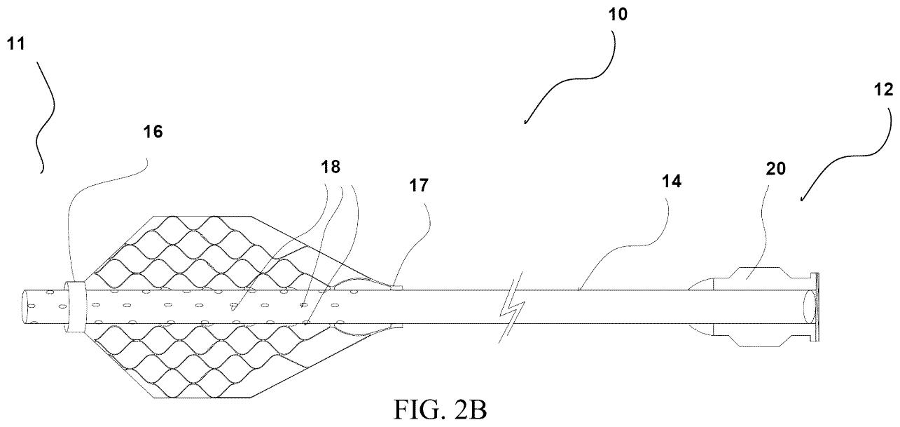

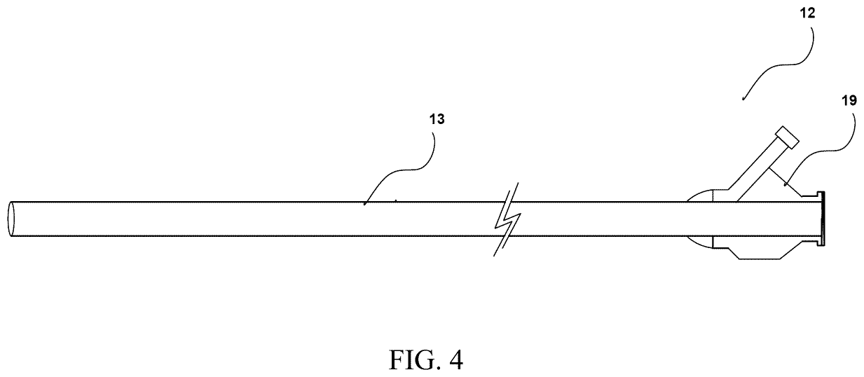

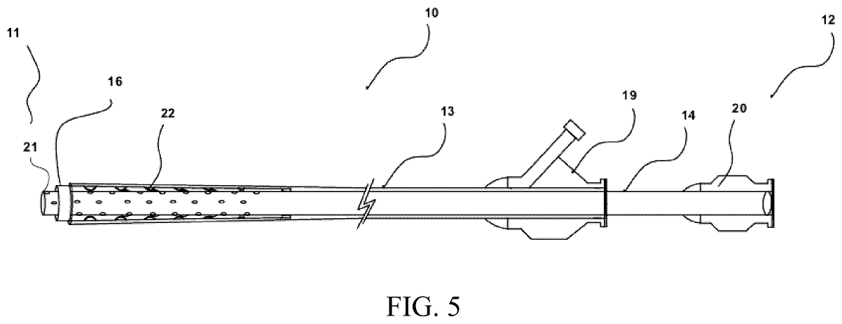

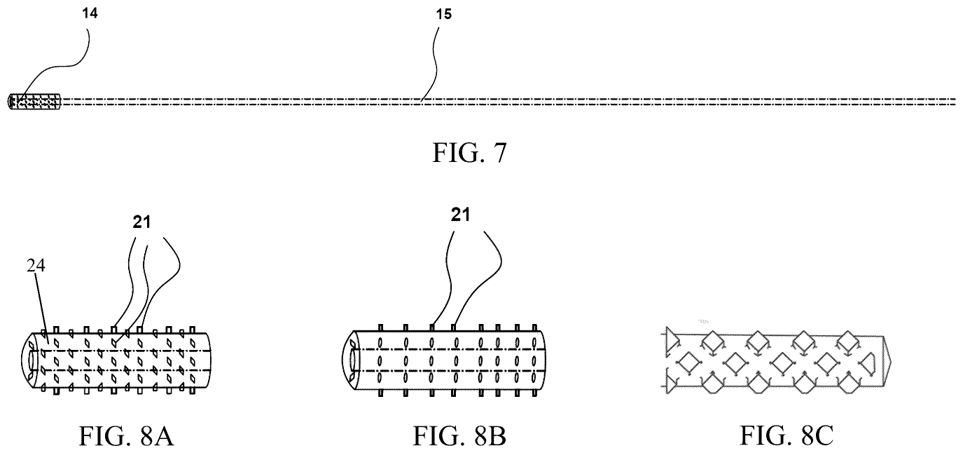

Referring now to the drawings wherein the showings are for the purpose of illustrating non-limiting embodiments of the disclosure only and not for the purpose of limiting same, FIGS. 1-12 illustrate several non-limiting embodiments of an endovascular medical device 10 are illustrated that can be used in the treatment of a bodily passageway that is obstructed by tissue mass such as a clot or thrombus.

Referring now to FIGS. 1-5, endovascular medical device 10 includes a) a capture tube 13, b) a second tubular body 14 that is slidably positioned within the capture tube and wherein second tubular body 14 extends beyond both the proximal and distal ends of the capture tube, c) an optional third tubular body 21 that is slidably positioned within second tubular body 14 and wherein a distal region of the third tubular body is located at or near the distal end of the second tubular body and can move outwardly from the distal end of the second tubular body 14, and d) an optional self-expandable stent or self-expandable porous structure 15.

The proximal end of capture tube 13 can optionally be attached to a y-arm luer 19. The y-arm luer can be connected to an aspiration device (not shown). The aspiration device can be configured to draw fluid and/or materials from the distal end or distal region of capture tube 13, the second tubular body 14, and/or the optional third tubular body and toward the proximal end or proximal region of capture tube 13, second tubular body 14, and/or the optional third tubular body. The y-arm luer can optionally be removable and attachable to capture tube 13.

The self-expanding stent or self-expandable porous structure is generally formed of nickel-titanium alloy (e.g., Nitinol™). Non-limiting configurations of the self-expanding stent or self-expandable porous structure are illustrated in FIGS. 1, 2A, 2B, 3A, and 3B.

The proximal end of the self-expanding stent or self-expandable porous structure is non-slidably connected to second tubular body 14. The type of connection arrangement is non-limiting (e.g., adhesive, solder, clamp, etc.). As illustrated in FIGS. 1, 2A, 2B and 5, the distal end of the self-expanding stent or self-expandable porous structure is connected to slidable ring 16 that is slidably positioned about a distal region of second tubular body 14. Slidable ring 16 is configured to move along the outer surface of second tubular body 14 as the self-expanding stent or self-expandable porous structure moves between the expanded and contracted positions.

As illustrated in FIGS. 1, 2A, 2B and 5, the region of the outer surface of second tubular body 14 located below the self-expanding stent or self-expandable porous structure includes one or more openings 18. One or more openings or holes 18 are configured to enable fluid and other material (e.g., clot fragments, etc.) to be drawn into and through one or more openings or holes 18 and into the interior cavity of the second tubular body when the aspiration device is activated. The number and/or size of one or more openings or holes 18 are non-limiting.

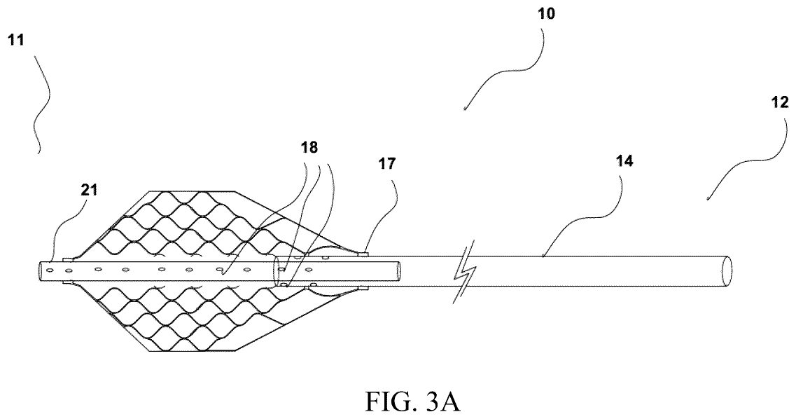

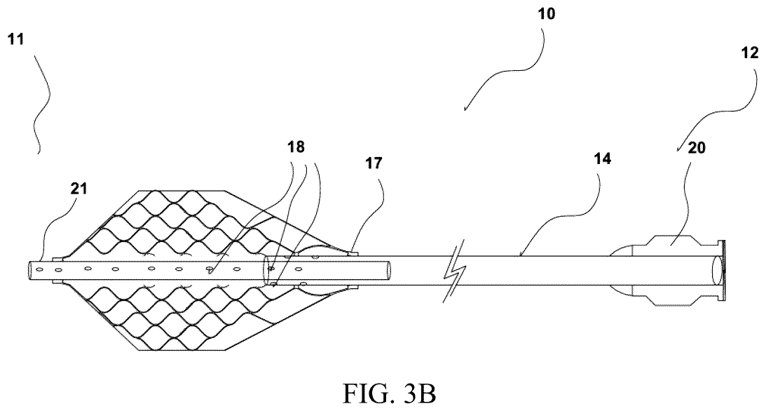

Referring now to FIGS. 3A and 3B, the endovascular medical device includes a third tubular member 21, wherein a proximal portion of the third tubular member is located in the cavity of second tubular body 14 and a distal portion of third tubular member 21 extends outwardly from the distal end of the second tubular body. A distal end of the self-expanding stent or self-expandable porous structure is non-slidably connected to a distal portion or distal end of third tubular member 21, and the proximal end of the self-expanding stent or self-expandable porous structure is non-slidably connected to an outer surface of second tubular member 14. Third tubular member 21 is configured to move within the interior cavity of second tubular body 14 as the self-expanding stent or self-expandable porous structure moves between the expanded and contracted positions.

As illustrated in FIGS. 3A and 3B, the region of the outer surface of third tubular body 21 that is located below the self-expanding stent or self-expandable porous structure includes one or more openings. The one or more openings or holes are configured to enable fluid and other material (e.g., clot fragments, etc.) to be drawn into and through the one or more openings or holes and into the interior cavity of third tubular body 21 when the aspiration device is activated. The fluid and other material drawn into the interior cavity of third tubular body 21 proceeds to flow toward the proximal end of the third tubular body and then drawn out of the endovascular medical device by the aspiration device. The number and/or size of the one or more openings or holes are non-limiting.

One non-limiting method of use of the endovascular medical device to partially or fully remove a clot from a body passageway comprises the steps of a) positioning the self-expanding stent or self-expandable porous structure fully or substantially inside the capture tube such that the self-expanding stent or self-expandable porous structure is in the unexpanded position; b) inserting a guidewire in the body passageway until the distal end of the guidewire is at least closely adjacent to the clot in the body passageway (e.g., within a few centimeters of the clot, at the proximal end of the clot, in the clot, beyond the distal end of the clot, etc.); c) introducing the endovascular medical device in the body passageway via the guidewire until the distal end of the endovascular medical device is at least closely adjacent to the proximal end of the clot; d) optionally removing the guidewire from the body passageway; e) activating aspiration device; f) deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway; g) moving one or more tubular bodies of the endovascular medical device to cause the at least partially expanded self-expanding stent or self-expandable porous structure to contact a portion of the clot and cause at least a portion of the clot to fracture and/or release from the body passageway and be drawn by the aspiration device into at least one of the tubular bodies (e.g., drawn into the distal end of at least one of the tubular bodies, drawing into and through one or more of the openings or holes in the surface of at least one of the tubular bodies, etc.); h) at least partially or fully retracting the at least partially expanded self-expanding stent or self-expandable porous structure into the cavity of the capture tube to cause the self-expanding stent or self-expandable porous structure to partially or fully contract, and wherein the fractured clot on the self-expanding stent or self-expandable porous structure is drawn from the self-expanding stent or self-expandable porous structure toward the proximal end of the capture tube by the aspiration device, and wherein the retracting of the self-expanding stent or self-expandable porous structure into the cavity of the capture tube optionally causes the self-expanding stent or self-expandable porous structure to grate against and at least partially fracture and/or remove the clot from the body passageway; i) optionally repeating steps f), g) and h) until the distal end of one or more of the tubular bodies are positioned distally to the distal end of the clot; j) optionally deploying the self-expanding stent or self-expandable porous structure such that at least a portion of the self-expanding stent or self-expandable porous structure is positioned forwardly of the distal end of the capture tube to enable the self-expanding stent or self-expandable porous structure to at least partially expand in the body passageway and moving the endovascular medical device to cause the at least partially expanded self-expanding stent or self-expandable porous structure to move across the former region of the clot so as to partially or fully remove remaining portions of the clot from the body passageway that remained in the body passageway after the completion of steps f), g), h) and i); k) at least partially or fully retracting the at least partially expanded self-expanding stent or self-expandable porous structure into the cavity of the capture tube to cause the self-expanding stent or self-expandable porous structure to partially or fully contract, and wherein fractured clots on the self-expanding stent or self-expandable porous structure are drawn from the self-expanding stent or self-expandable porous structure toward the proximal end of the capture tube by the aspiration device; and l) remove the endovascular medical device from the body passageway.

One modified method for using the endovascular medical device to partially or fully remove a clot from a body passageway comprises the steps of a guide sheath and guide wire being used to position the distal end of the endovascular medical device at a location that is distal to the distal end of the clot; thereafter, the self-expanding stent or other type of self-expanding porous structure can be deployed and partially or fully expanded. The deployed self-expanding stent or other type of self-expanding porous structure is moved toward the proximal end of the clot while the aspiration device is activated, and wherein during the movement of the self-expanding stent or other type of self-expanding porous structure is moved toward the proximal end of the clot, the self-expanding stent or other type of self-expanding porous structure can be retracted into the capture tube and then redeployed one or more times.

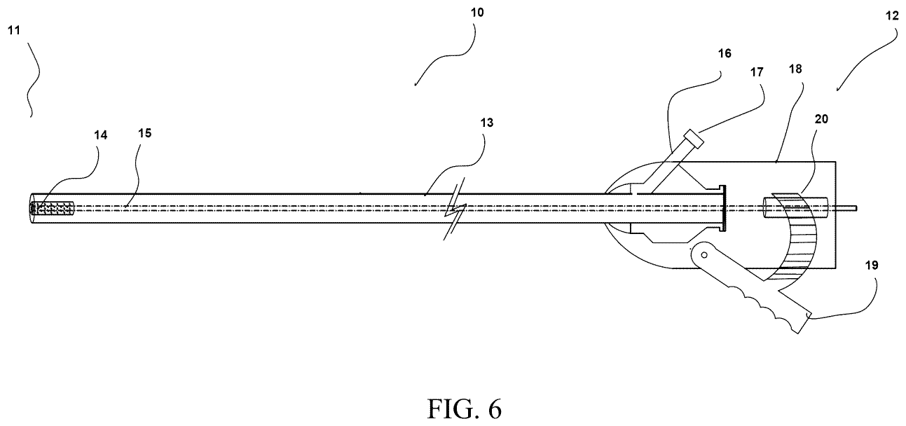

Referring now to FIGS. 6-10B, there is illustrated an endovascular medical device 10 that can be used to partially or fully remove a clot or thrombus (not shown) in a body passageway.

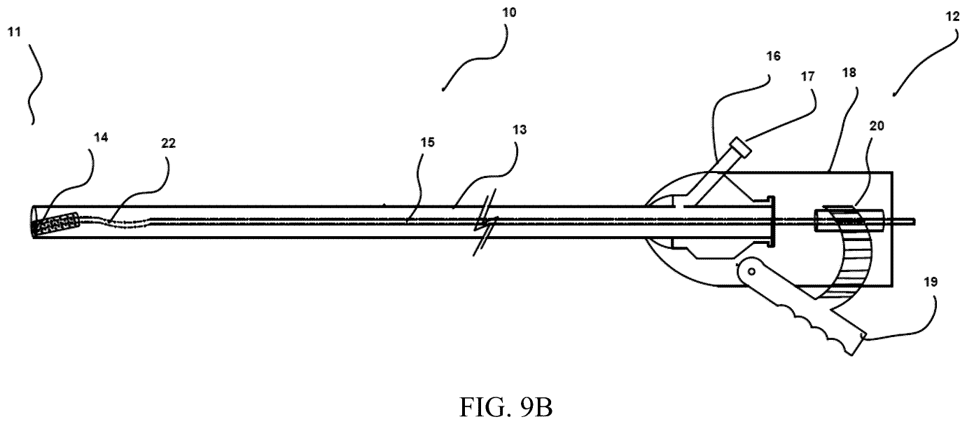

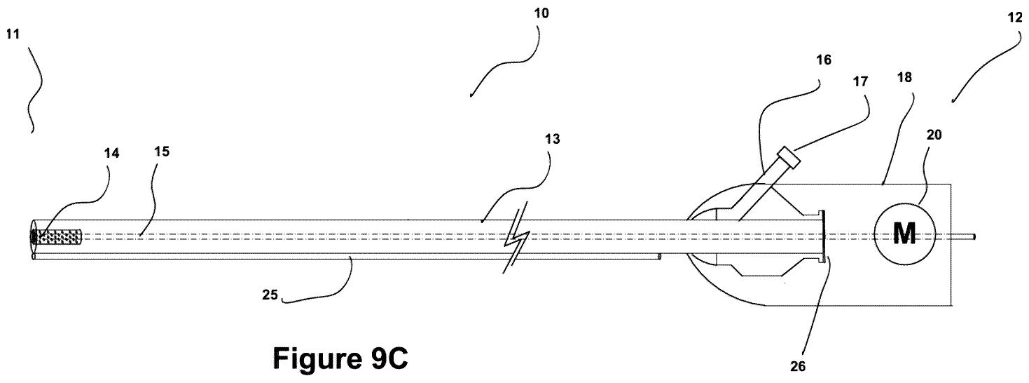

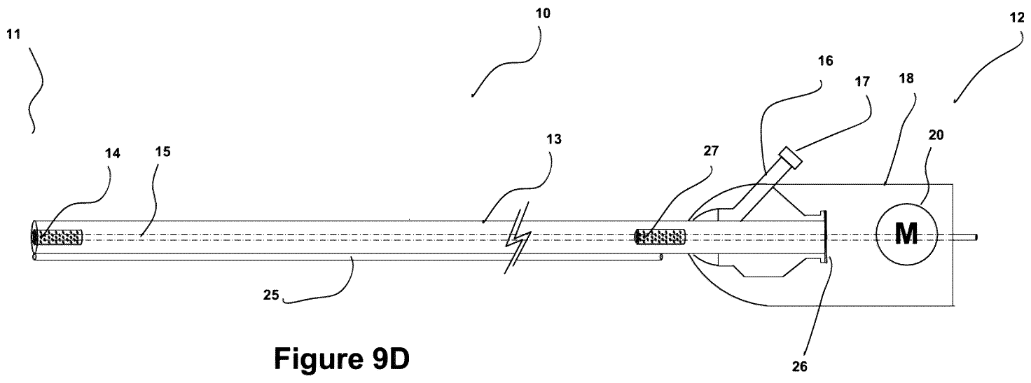

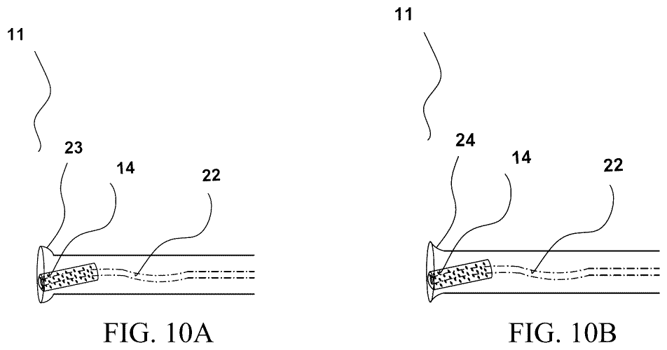

The endovascular medical device 10 has a distal end 11 and a proximal end 12. The endovascular medical device comprises a catheter 13, a device inner shaft 15 that is at least partially positioned in the cavity of the catheter and runs at least partially or fully the length of catheter 13, and a fragmenter 14 positioned at the front end or front end portion of the device shaft. The inner shaft 15 not limited in shape, and in one non-limiting configuration the inner shaft is a tubular structure through which a guide wire may be passed through the entire length of the device. In another non-limiting configuration, the inner shaft can be a solid wire. Catheter 13 can be formed of one or more hollow tubular bodies. As illustrated in FIGS. 6 and 9B, catheter 13 is formed of a single tube. As illustrated in FIG. 9C, the catheter can include a guide wire tube 25.

The proximal end of the endovascular medical device 10 includes a handle 18 connected to the proximal end of catheter 13. The length, shape, and/or materials of catheter 13 and handle are non-limiting. The handle is configured for use by a medical practitioner to insert and/or orient the catheter in the body passageway of the patient. The device shaft runs about 100% of the longitudinal length of catheter 13 located in the body passageway of the patient.

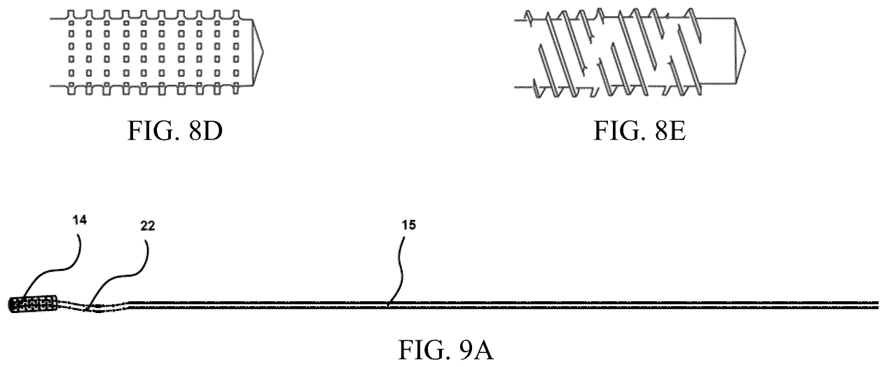

The fragmenter 14 includes a main housing 24 and a non-smooth surface that is formed of non-smooth surface structures 21 (e.g., ribs, spikes, bumps, undulations, channels, slots, recesses, barbs, pins, blades, surface extensions, fluted surface extensions, etc.) that extend outwardly from the top surface of the main housing. The number and shape of non-smooth surface structures are non-limiting. In one non-limiting embodiment, all of the non-smooth surface structures 21 are oriented symmetrically with one another as illustrated in FIGS. 8B, 8C and 8D. In another non-limiting embodiment, a plurality of non-smooth surface structures 21 are oriented non-symmetrically with one another as illustrated in FIG. 8A. As illustrated in FIGS. 8A and 8B, the non-smooth surface structures 21 can optionally be generally pin-shaped. As illustrated in FIGS. 8C and 8D, the non-smooth surfaces can be cubic-shaped or cuboid-shaped. As illustrated in FIG. 8E, the non-smooth surface structures 21 are spiral-shaped. As can be appreciated, the non-smooth surface structures 21 illustrated in FIG. 8E can be continuous to form a threaded-shaped non-smooth surface. As can be appreciated, non-smooth surface structures 21 can have a variety of different shapes and sizes. As can also be appreciated, all of non-smooth surface structures 21 can have the same shape and/or size, or a plurality of the non-smooth surface structures 21 can have different shapes and/or sizes. Generally, non-smooth surface structures 21 cover 20-80% (and all values and ranges therebetween) of the top surface of the fragmenter. The maximum height of a plurality or all of the non-smooth surface structures 21 is generally less than 15% (e.g., 0.01-14.99% and all values and ranges therebetween) the inner diameter of the main catheter housing 13. The main housing has a generally cylindrical shape; however, other shapes can be used.

The device shaft is illustrated as interconnected to the handle to enable the device shaft to be reciprocated and/or rotated in the catheter to thereby cause the reciprocation and/or rotation of the fragmenter in the body passageway. The movement of the fragmenter relative to the clot in the body passageway causes one or more of the non-smooth surface structures 21 to contact the clot to thereby scrape off or fragment a portion or all of the clot in the body passageway. In one non-limiting embodiment, the handle includes a handle lever 19 that is interconnected to a rack and pinion mechanism 20. The squeezing and releasing of the handle lever enables a medical practitioner to cause the device shaft to reciprocate and/or rotate during a clot removal procedure. In another non-limiting embodiment, the handle houses a motor that is capable of rotating the inner shaft. The motor, when used, is configured to rotate the inner shaft, which in turn rotates the fragmenter. In another non-limiting embodiment, the motor is configured to rotate the inner shaft in the clockwise and/or counterclockwise direction. The rotational speeds of the inner shaft and/or fragmenter can range from 1-5000 revolutions per minute (rpm) (and all values and ranges therebetween).

Referring now to FIGS. 9A and 9B, the device shaft can include a non-linear profile (e.g., wavy profile, curved profile, etc.) at the distal region 22 of the device shaft. Such a non-linear device shaft profile causes the fragmenter to wobble within the catheter body or extended in the body passageway to facilitate in causing the non-smooth surface structures 21 to contact the clot in the body passageway. In one non-limiting embodiment, the non-linear shape of the distal portion of the device shaft causes the fragmenter to rotate in an elliptical motion around the central axis of the catheter as well as rotation around the fragmenter's own central axis.

Referring now to FIGS. 6, 9B and 9C, an aspirator system can be connected to an aspiration port 17 in the y-arm luer 16 that is connected to the handle 18. The aspirator system draws fragments of the clot that have been removed from the clot in the body passageway as the non-smooth surface structures 21 of the fragmenter contact the clot into the catheter. The straight arm of the y-arm luer can optionally have a hemostasis valve 26 through which the proximal end of the device shaft proximally exits such that the hemostasis valve does not excessively obstruct or otherwise interfere with the rotation and/or reciprocation of the device shaft while at the same time the hemostasis valve inhibits or prevent air and/or fluid leakage. In one non-limiting embodiment, two or more fragmenters can optionally be located along the length of the inner shaft 15 and withing the catheter body 13. As shown in FIG. 9D there are two fragmenters 14 and 27.

Generally, the device shaft is configured such that the distal end of the fragmenter is located at or spaced slightly inwardly from the distal end of the catheter prior to the reciprocation and/or rotation of the fragmenter.

In operation, the endovascular medical device can be used to partially or fully remove a clot from a body passageway. The method of operation includes a) navigating a guide wire to the treatment site within the vasculature; b) inserting the distal end or distal portion of the catheter on the proximal end of the guide wire; c) moving the catheter over the guide wire until the distal end of the catheter is located at or near the treatment site; d) connecting an aspiration system to the aspiration port on the handle and/or catheter (which step can occurred at any time prior to the reciprocation and/or rotation of the fragmenter); e) activating the aspiration system to draw fluid to from the distal portion of the catheter to the proximal portion of the catheter (which step typically occurs prior to the beginning of the reciprocation and/or rotation of the fragmenter); f) moving the distal end of the catheter to or closely adjacent to the proximal end of the clot if not already positioned at the clot; g) optionally retracting the guide wire from the catheter after the catheter has been positioned at or closely adjacent to the clot; h) causing the fragmenter to be reciprocated and/or rotated to cause at least a portion of the fragmenter to engage at least a portion of the clot to cause partial or full fragmentation and/or removal of the clot from the vasculator; and i) removing the catheter, device shaft, and fragmenter from the vasculaturer once the desired amount of the clot has been partially or fully removed from the vasculature.

The catheter can optionally be moved forwardly after a portion of a clot has been removed and step h) can be repeated to further remove additional clot from the body passageway. The catheter can be moved forwardly one or more times to partially or fully remove the clot along the full length of a long clot.

When the fragmenter is caused to reciprocate, all or a portion of the fragmenter extends forwardly of the distal end of the catheter to enable at least a portion of the fragmenter to contact the clot in the body passageway.

Referring now to FIGS. 10A and 10B, the distal tip of the catheter can optionally be configured to radially expand to form a funnel shaped opening for better capture of clot fragments produced by the contact of the fragmenter with the clot.

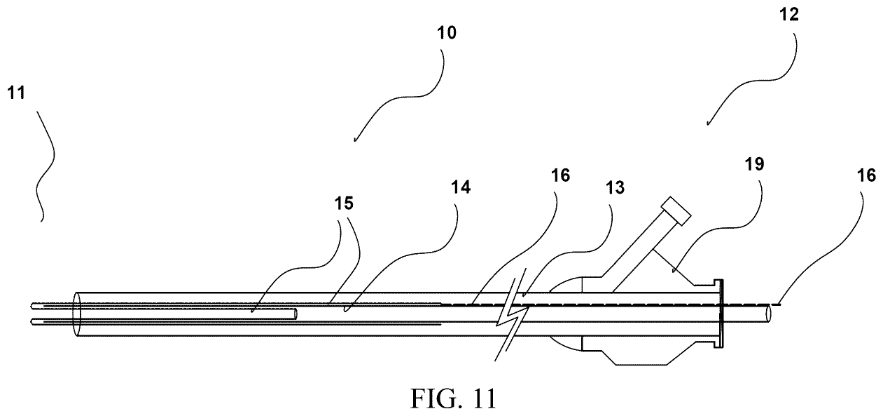

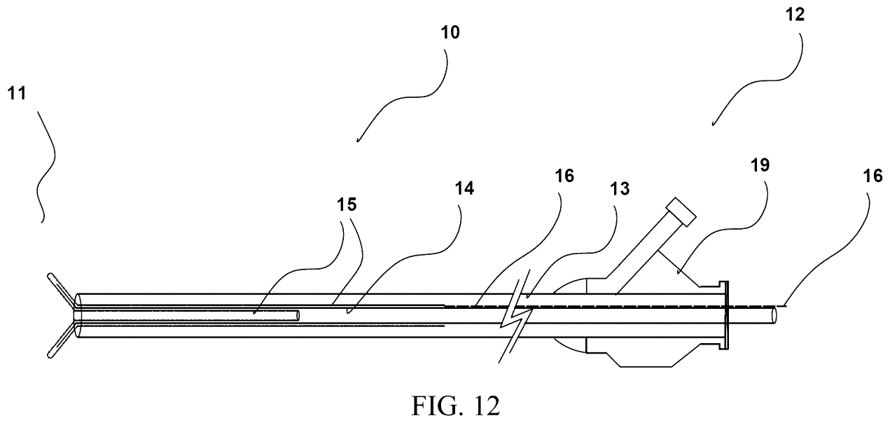

Referring now to FIGS. 11 and 12, there is illustrated another non-limiting embodiment of the endovascular medical device 10 in accordance with the present disclosure.

The endovascular medical device 10 includes a catheter formed of a first outer tubular structure 13 (or catheter body), a second tubular structure 14, and a clot engagement material 15. Second tubular structure 14 is secured in position relative to the first tubular structure 13 such that the second tubular structure does not move longitudinally within the first tubular structure. At least a portion of the distal portion of the second tubular structure extends beyond the distal end of the first tubular structure. Generally, a majority (e.g., 55-98% and all values and ranges therebetween) of the second tubular structure 14 is positioned within the first tubular structure 13. Clot engagement material 15 is generally a mesh or braided material; however, other materials can be used. Clot engagement material 15 can be folded (e.g., folded edge-to-edge, etc.) to form an incomplete tubular structure within first tubular structure 13. A tether wire 16 can be connected to the front end of clot engagement material 15 to enable the clot engagement material located within the interior of the second tubular structure to be pulled along the outer surface of the second tubular structure toward the proximal end of the second tubular structure during a clot removal procedure.

The proximal portion of the first tubular structure includes a y-arm luer 19 with an optional hemostasis valve. An aspirator system to draw fluid into the catheter can be connected to the y-arm luer.

Although not shown, it will be appreciated that the distal end of second tubular structure 14 can be positioned beyond the distal end of the clot. The distal portion of second tubular structure 14 is illustrated as being flared outwardly. During clot removal, the aspiration system is activated prior to the pulling of the clot engagement material 15 by tether wire 16 over the top surface of second tubular structure 14 and toward the proximal end of the second tubular structure. While clot engagement material 15 is moved over the top surface of second tubular structure 14, first tubular structure 13 is moved from the distal end of the clot to the proximal.

In another non-limiting embodiment of endovascular medical device 10 (not shown in detail), endovascular medical device 10 includes a catheter formed of a first outer tubular structure 13 (or catheter body), a second tubular structure 14, and a third tubular structure, and a clot engagement material 15. The third tubular structure is slidable positioned within the first tubular structure. A majority (e.g., 60-100% and all values and ranges therebetween) of the second and third tubular structures are positioned within the longitudinal cavity of the first tubular structure. Clot engagement material 15 is generally a mesh or braided material; however, other materials can be used. Clot engagement material 15 can be folded (e.g., folded edge-to-edge, etc.) to form an incomplete tubular structure within the first tubular structure.

The third tubular structure has a proximal end and a distal end, wherein the distal end or distal end portion is connected to an end or end portion of the clot engagement material. The clot engagement material can be partially or fully wrapped around the outer surface of the distal portion of the third tubular structure. Prior to deployment of the clot engagement material, the distal ends of the second and third tubular structures are aligned or substantially aligned. The clot engagement material can be inverted proximally over the distal end portion of the second tubular structure. The first tubular structure is placed over the inverted clot engagement material. The distal end of the first tubular structure, prior to deployment of the clot engagement material, is also aligned or substantially aligned with the distal ends of the second and third tubular structures. Such an arrangement forms a structure in which the inverted clot engagement material lies in between the first tubular structure and the second tubular structure and the separation slit of the clot engagement material runs longitudinally along a portion or all of the longitudinal length of the three tubular structures.

The first tubular structure includes a y-arm luer 19 with an optional hemostasis valve. The y-arm is fluidly connected or interconnected to the proximal end portion of the second tubular structure. An aspirator system to draw fluid into the catheter can be connected to the y-arm luer. The third tubular structure generally protrudes proximally through the hemostasis valve. A luer can optionally be attached to the proximal end or end region of the third tubular structure and be located proximal to the y-arm that is connected to the second tubular structure.

The first, second, and third tubular structures are aligned or substantially aligned with one another along the longitudinal axis of the catheter.

The distal ends of the third tubular structure and the clot engagement material are distal or located forwardly to the distal edge of the second tubular structure. The second tubular structure and the clot engagement material can optionally be bonded to each other.

Referring now to FIGS. 13B and 13C, there is method for removing a clot using the endovascular medical device wherein a) the first tubular structure is navigated over a guide wire to the treatment site and the distal end of the first tubular structure is placed at or closely adjacent to the distal end of the clot in the body passageway, b) the third tubular structure is pulled toward the proximal end of the first tubular structure while the distal end of the first tubular structure is moved toward the proximal end of the clot and into the clot. The second tubular structure connected to the interior of the first tubular structure remains in position relative to the first tubular structure as the third tubular structure is retracted rearwardly from the first tubular structure. The opposite movements of the first and third tubular structures causes the clot engagement material to move distally over the top of the second tubular structure while the clot engagement material moves proximally inside the second tubular structure. Such movement of the clot engagement material along the top surface of the second tubular structure and then into the interior of the second tubular structure causes the clot that engages the clot engagement material to be captured by the moving clot engagement material as the clot engagement material moves about the end of the second tubular structure and then into the interior of the second tubular structure. While the first and third tubular structure are moved in opposite directions to one another, the aspiration system is used to facilitate in drawing the clot into the interior of the second tubular structure.

The distal portion of the second tubular structure (and optionally also the first tubular structure) can optionally be configured to be outwardly flared. Such flaring can optionally be used to cause the clot material, as it is being drawn into the interior of the tube, to invert over itself to thereby facilitate in removing and capturing the clot material.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the preferred embodiments. These and other modifications of the preferred embodiments as well as other embodiments of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

What is claimed:

1. An endovascular device that includes an outer tubular catheter body, an inner tubular body that includes one or more fragmenters; at least one of said fragmenters is located at a distal end said inner tubular body and can be positionable within a distal end of said outer tubular catheter body when said inner tubular body is at least partially retracted in said outer tubular catheter body; said inner tubular body and said one or more fragmenters movable relative to said outer tubular catheter body by a hand-operated mechanical arrangement and/or by a motor arrangement; said outer tubular catheter body includes a luer connector.

2. The endovascular device as defined in claim 1, wherein said inner tubular body and said one or more fragmenters are configured to be rotatable relative to the outer tubular catheter body.

3. The endovascular device as defined in claim 1, wherein said inner tubular body and said one or more fragmenters are configured to reciprocate relative to the outer tubular catheter body.

4. The endovascular device as defined in claim 1, wherein said inner tubular body and said one or more fragmenters are configured to vibrate relative to the outer tubular catheter body.

5. The endovascular device as defined in claim 1, wherein said inner tubular body and said one or more fragmenters are configured to radiate electromagnetic waves relative to the outer tubular catheter body.
The endovascular device as defined in claim 1, wherein a proximal end of said luer connector includes a hemostasis valve through which a proximal end said inner tubular body can extend outwardly therefrom.

6. The endovascular device as defined in claim 1, wherein said inner tubular body is hollow and is configured to allow a guide wire to pass there through said inner tubular body.

7. The endovascular device as defined in claim 1, wherein said inner tubular body is hollow and is configured to allow fluid to flow through said hollow tubular body; said fluid includes one or more of saline solution, water, clot dissolving thrombolytic agent, and/or anti-clotting agent.

8. The endovascular device as defined in claim 1, wherein said fragmenter includes a non-smooth outer surface that includes one or more non-smooth surface structures; said one or more non-smooth surface structures include one or more structures selected from the group consisting of ribs, spikes, bumps, undulations, channels, slots, recesses, barbs, pins, blades, surface extensions, and fluted surface extensions.

9. The endovascular device as defined in claim 1, wherein said inner tubular body is partially or fully formed of 1-16 wires.

10. The endovascular device as defined in claim 1, wherein said inner tubular body is partially or fully formed of a plurality of wires that are braided together; said inner tubular body is flexible.

11. An endovascular medical device for the treatment of a bodily passageway that is obstructed by tissue mass such as a clot or thrombus; said endovascular medical device comprises a catheter, a device shaft at least partially positioned in the cavity of the catheter and running at least partially the length of the catheter, and a fragmenter positioned at the front end or front end portion of the device shaft.

12. The endovascular medical device as defined in claim 11, further including a handle that is connected to a proximal portion of said catheter; said handle configured to cause a) rotation of said device shaft, and/or b) reciprocation of said device shaped along a longitudinal axis of said catheter.

13. The endovascular medical device as defined in claim 11, wherein said fragmenter includes a main housing and a plurality of non-smooth surface structures that extend outwardly and/or inwardly from said top surface of the main housing.

14. The endovascular medical device as defined in claim 11, wherein said device shaft includes a non-linear profile at a distal region of the device shaft.

15. The endovascular medical device as defined in claim 11, further including an aspirator system that is configured to draw material into said distal end of said catheter.

16. The endovascular medical device as defined in claim 11, wherein a distal end portion of said catheter is configured to radially expand to form a funnel-shaped opening.

17. An endovascular medical device for the treatment of a bodily passageway that is obstructed by tissue mass such as a clot or thrombus; said endovascular medical device comprising a) a catheter that includes first, second, and third tubular structures; said first and third tubular structures are slidably movable with respect to one another; and wherein said second and third tubular structure is positioned in the longitudinal cavity of said first tubular structure, and said third tubular structure is positioned in the longitudinal cavity of said second tubular structure, and b) a clot engagement material.

18. The endovascular medical device as defined in claim 17, wherein said clot engagement material is a mesh or braided material.

19. The endovascular medical device as defined in claim 17, wherein said clot engagement material is folded edge-to-edge to form an incomplete tubular structure within said first tubular structure.

20. The endovascular medical device as defined in claim 17, wherein a distal end or distal end portion of said third tubular structure is connected to an end or end portion of said clot engagement material.

21. The endovascular medical device as defined in claim 17, wherein said second tubular structure is oriented relative to said first tubular structure to not move or substantially not move along the longitudinal axis of said first tubular structure.

22. The endovascular medical device as defined in claim 17, wherein said second tubular structure is connected to an interior of said first tubular structure.

23. The endovascular medical device as defined in claim 17, wherein said clot engagement material is partially or fully wrapped around an outer surface of at least a portion of said third tubular structure.

24. The endovascular medical device as defined in claim 17, wherein a) said first tubular structure is navigated over a guide wire to a treatment site and a distal end of said first tubular structure is placed at or closely adjacent to a proximal end of a clot in a body passageway, and b) said third tubular structure is pulled toward a proximal end of said first tubular structure while a distal end of said first tubular structure is move toward and into said clot.

25. The endovascular medical device as defined in claim 17, wherein said distal end portion of said second tubular structure is expandable to enable said distal end portion to flare outwardly.

26. The endovascular medical device as defined in claim 17, wherein said distal edge of said first tubular structure is connected to a distal edge of said third tubular structure; said distal portion of said first tubular structure formed of a soft flexible material that allows said first tubular structure to collapse over itself and invert as said first tubular structure is pulled into said third tubular structure.

27. An endovascular medical device for the treatment of a bodily passageway that is obstructed by tissue mass such as a clot or thrombus; said endovascular medical device comprises a) a catheter that includes first and second tubular structures; and wherein said first and second tubular structures are not slidably movable with respect to one another; and wherein said second tubular structure is positioned in the longitudinal cavity of said first tubular structure, and b) a clot engagement material that is at least partially positioned in an interior cavity of said second tube; and wherein said clot engagement material is configured to be drawn from said interior cavity of said second tube, about a distal end of said second tube and between an outer surface of said second tube and an inner cavity surface of said first tube.

28. The endovascular medical device as defined in claim 27, wherein said clot engagement material is a mesh or braided material.

29. The endovascular medical device as defined in claim 27, wherein said clot engagement material is folded edge-to-edge to form an incomplete tubular structure within said first tubular structure.

30. The endovascular medical device as defined in claim 27, wherein a tether wire is connected to a front end or end portion of said clot engagement material; said tether wire located between an outer surface of said second tube and an inner cavity surface of said first tube and extending to a proximal end of said first tube.

31. The endovascular medical device as defined in claim 27, wherein said second tubular structure is oriented relative to said first tubular structure to not move or substantially not move along the longitudinal axis of said first tubular structure.

32. The endovascular medical device as defined in claim 27, wherein said second tubular structure is connected to an interior of said first tubular structure.

33. The endovascular medical device as defined in claim 27, wherein a) said first tubular structure is navigated over a guide wire to a treatment site and a distal end of said second tubular structure is placed beyond a distal end of a clot in a body passageway, b) an aspiration system is activated prior to pulling of said clot engagement material over an outer surface of said second tube, c) pulling said clot engagement material proximally along said outer surface of said second tubular structure such that said clot engagement material is pulled distally in said interior of the second tubular structure toward the distal end of said second tubular structure and then about a distal end of said second tubular structure and then along said outer of the second tubular structure toward a proximal end of said second tubular structure, and d) moving said distal end of the second tubular structure from a distal end of said clot to a proximal end of said clot while said clot removal material is drawn about said distal end of said second tubular structure.

34. The endovascular medical device as defined in claim 27, wherein said distal end portion of said second tubular structure is expandable to enable said distal end portion to flare outwardly.

35. The endovascular medical device as defined in claim 27, wherein said distal end portion of said second tubular structure is oblique cut to form a larger opening at said distal end of said second tubular structure.

36. An endovascular medical device for the treatment of a bodily passageway that is obstructed by tissue mass such as a clot or thrombus; said endovascular medical device comprises a) a capture tube, b) a second tubular body that is slidably positioned within said capture tube and wherein the second tubular body optionally extends beyond both a proximal and distal ends of said capture tube, c) an optional third tubular body that is slidably positioned within the second tubular body and wherein a distal region of said third tubular body is located at or near a distal end of said second tubular body and can move outwardly from said distal end of said second tubular body, and d) a self-expandable stent or self-expandable porous structure.

37. The endovascular medical device as defined in claim 36, wherein a proximal end of said capture tube is attached to a y-arm luer, and herein said y-arm luer is connected to an aspiration device.

38. The endovascular medical device as defined in claim 36, wherein said self-expanding stent or self-expandable porous structure is formed of a nickel-titanium alloy.

39. The endovascular medical device as defined in claim 36, wherein a proximal end of said self-expanding stent or self-expandable porous structure is non-slidably connected to said second tubular body.

40. The endovascular medical device as defined in claim 36, wherein said self-expanding stent or self-expandable porous structure is non-slidably connected at a proximal end of said self-expanding stent or self-expandable porous structure to said second tubular member, and a distal end of said self-expanding stent or self-expandable porous structure is connected to a slidable ring that is slidably positioned about a distal region of said second tubular body.

41. The endovascular medical device as defined in claim 36, wherein a region of an outer surface of said second tubular body located below said self-expanding stent or self-expandable porous structure includes one or more openings.

42. The endovascular medical device as defined in claim 36, wherein a proximal portion of said third tubular member is located in a cavity of said second tubular body and a distal portion of said third tubular member extends outwardly from a distal end of said second tubular body; and wherein a distal end of said self-expanding stent or self-expandable porous structure is non-slidably connected to a distal portion or distal end of said third tubular member, and a proximal end of said self-expanding stent or self-expandable porous structure is non-slidably connected to an outer surface of said second tubular member; and wherein said third tubular member is configured to move within an interior cavity of said second tubular body.

43. The endovascular medical device as defined in claim 42, wherein a region of an outer surface of said third tubular body located below said self-expanding stent or self-expandable porous structure includes one or more openings.

44. The endovascular medical device as defined in claim 36, wherein said capture tube can include a side opening; said side opening optionally includes an elastic septum that allows said second tubular body to pass therethrough, but will form a seal around said second tubular body.

45. A method of using the endovascular medical device as defined in claim 36 comprising the steps of a) positioning said self-expanding stent or self-expandable porous structure fully or substantially inside said capture tube such that said self-expanding stent or self-expandable porous structure is in an unexpanded position; b) inserting a guidewire in a body passageway until a distal end of said guidewire is at least closely adjacent to said clot in said body passageway; c) introducing said endovascular medical device in said body passageway via said guidewire until a distal end of said endovascular medical device is at least closely adjacent to said proximal end of the clot; d) optionally removing said guidewire from said body passageway; e) activating an aspiration device; f) deploying said self-expanding stent or self-expandable porous structure such that at least a portion of said self-expanding stent or self-expandable porous structure is positioned forwardly of said distal end of the capture tube to enable said self-expanding stent or self-expandable porous structure to at least partially expand in said body passageway; g) moving one or more tubular bodies of said endovascular medical device to cause said at least partially expanded self-expanding stent or self-expandable porous structure to contact a portion of said clot and cause at least a portion of said clot to fracture and/or release from said body passageway and be drawn by said aspiration device into at least one of said tubular bodies; h) at least partially or fully retracting said at least partially expanded self-expanding stent or self-expandable porous structure into said cavity of said capture tube to cause said self-expanding stent or self-expandable porous structure to partially or fully contract, and wherein fractured clots on the self-expanding stent or self-expandable porous structure are drawn from said self-expanding stent or self-expandable porous structure toward said proximal end of said capture tube by said aspiration device, and wherein said retracting of said self-expanding stent or self-expandable porous structure into said cavity of said capture tube optionally causes said self-expanding stent or self-expandable porous structure to grate against and at least partially fracture and/or remove said clot from said body passageway; i) optionally repeating steps f), g) and h) until a distal end of one or more of said tubular bodies are positioned distally to a distal end of said clot; j) optionally deploying said self-expanding stent or self-expandable porous structure such that at least a portion of said self-expanding stent or self-expandable porous structure is positioned forwardly of said distal end of said capture tube to enable said self-expanding stent or self-expandable porous structure to at least partially expand in said body passageway, and thereafter optionally moving said endovascular medical device to cause said at least partially expanded self-expanding stent or self-expandable porous structure to move across a former region of said clot to partially or fully remove remaining portions of said clot from said body passageway that remained in said body passageway after completion of steps f), g), h) and i); k) at least partially or fully retracting said at least partially expanded self-expanding stent or self-expandable porous structure into said the cavity of said capture tube to cause said self-expanding stent or self-expandable porous structure to partially or fully contract, and wherein fractured clots on said self-expanding stent or self-expandable porous structure are optionally drawn from said self-expanding stent or self-expandable porous structure toward said proximal end of said capture tube by said aspiration device; and l) removing said endovascular medical device from said body passageway.

46. A method of using the endovascular medical device as defined in claim 36 comprising the steps of positioning a guide sheath and guide wire to a distal end of a clot, positioning a distal end of said endovascular medical device at a location that is distal to said distal end of the clot, causing said self-expanding stent or other type of self-expanding porous structure to be deployed and partially or fully expanded, moving the deployed and partially or fully expanded self-expanding stent or other type of self-expanding porous structure toward a proximal end of said clot while an aspiration device is activated.
